(19) United States
(12) Patent Application Publication
(10) Pub. No.: US 2025/0339075 A1
(43) Pub. Date: Nov. 6, 2025

(54) BIOLOGICAL SENSOR

(71) Applicant: NITTO DENKO CORPORATION, Ibaraki-shi, Osaka (JP)

(72) Inventors: Sota KONDO, Ibaraki-shi, Osaka (JP); Keine NISHIYAMA, Ibaraki-shi, Osaka (JP); Chiharu YANO, Ibaraki-shi, Osaka (JP)

(21) Appl. No.: 18/869,866

(22) PCT Filed: May 31, 2023
(86) PCT No.: PCT/JP2023/020226

§ 371 Date: Nov. 27, 2024

(30) Foreign Application Priority Data
Jun. 3, 2022 (JP) 2022-090963

(51) Int. Cl. A61B 5/257

(52) U.S. Cl. CPC A61B 5/257

(57) ABSTRACT

A biological sensor according to the present invention includes a sensor body configured to obtain biological information; an electrode having adhesiveness and connected to the sensor body; a first layer member including a housing space in which the sensor body is housed, the electrode being disposed on a lower surface of the first layer member; and a second layer member that is attached to the lower surface of the first layer member so as to expose the electrode and cover the sensor body. A thickness of the electrode is 15 μm or more, an area of the electrode is from 2.0 cm² through 5.0 cm², and a tack force of the electrode is 60 gf/Φ5 mm or more. In a bottom view, a covering percentage of the electrode with respect to the first layer member is from 40% through 90%.

### TECHNICAL FIELD

The present invention relates to a biological sensor.

### BACKGROUND ART

A biological sensor configured to perform measurement of biological information, such as an electrocardiogram waveform, a pulse wave, an electroencephalogram, an electromyogram, or the like, is used in medical institutions, such as a hospital, a clinic, and the like, nursing facilities, ones' homes, and the like. The biological sensor includes a biological electrode configured to obtain biological information of subjects by contact with their living body. When measuring such biological information, the biological sensor is attached to skin of a subject, and an electric signal of the biological information is obtained by the biological electrode. As a result, measurement of the biological information is performed.

As such a biological sensor, for example, a biological sensor including a sensor body, an electrode, a first layer member, and a second layer member is disclosed. In this biological sensor, the first layer member is formed by stacking a cover on an upper sheet and is configured to house the sensor body, and the second layer member is attached to a surface of the first layer member on the living body side and is formed such that the sensor body is disposed and the electrode is exposed (see, for example, PTL 1).

This biological sensor obtains biological information by attaching, to skin, a first adhesive layer provided on a surface of the first layer member facing the living body and a second adhesive layer provided on a surface of the second layer member facing the living body, and contacting the electrode attached to the first adhesive layer with the skin.

### CITATION LIST

### Patent Literature

PTL 1: Japanese Patent No. 6947955

### SUMMARY OF THE INVENTION

### Technical Problem

PTL 1 does not study the thickness and the area of the electrode, the contact impedance with the skin, and the ease of peeling off from the surface of the living body. When the thickness or the area of the electrode is increased, the contact impedance with the surface of the living body decreases, and the noise of the detected electric signal is suppressed. However, there is a possibility that an attachment performance to the surface of the living body, such as, for example, skin of a subject, is degraded and likely to cause peeling.

A biological sensor is often used for a long time in a state of being attached to the surface of the living body, such as skin or the like, and is required to detect biological information with high accuracy. Therefore, in order for the biological sensor to stably obtain an electric signal indicating biological information from the surface of the living body, such as skin or the like, with high accuracy for a long time, it is desirable that the biological sensor can be maintained in a state of being stably attached to the surface of the living body while suppressing generation of noise of the detected electric signal.

In one aspect of the present invention, it is an object to provide a biological sensor that can suppress the generation of noise during use and can be stably attached to the living body.

### Solution to the Problem

One aspect of the biological sensor according to the present invention includes: a sensor body configured to obtain biological information; an electrode having adhesiveness and connected to the sensor body; a first layer member including a housing space in which the sensor body is housed, the electrode being disposed on a lower surface of the first layer member; and a second layer member that is attached to the lower surface of the first layer member so as to expose the electrode and cover the sensor body. A thickness of the electrode is 15 μm or more, an area of the electrode is from 2.0 cm² through 5.0 cm², and a tack force of the electrode is 60 gf/Φ5 mm or more. In a bottom view, a covering percentage of the electrode with respect to the first layer member is from 40% through 90%.

### Advantageous Effects of the Invention

According to one aspect of the present invention, the biological sensor can suppress the generation of noise during use and can be stably attached to the living body.

### BRIEF DESCRIPTION OF THE DRAWINGS

FIG. 1 is a perspective view illustrating an entire configuration of a biological sensor according to an embodiment of the present invention.

FIG. 2 is a plan view illustrating examples of parts of the biological sensor.

FIG. 3 is a longitudinal cross-sectional view of the biological sensor taken along the line I-I in FIG. 1.

FIG. 4 is a bottom view of the biological sensor of FIG. 1.

FIG. 5 is an explanatory view illustrating the biological sensor of FIG. 1 attached to the chest of a living body.

### DESCRIPTION OF THE EMBODIMENTS

In the following, embodiments of the present invention will be described in detail. For ease of understanding to the description, the same components in the drawings are denoted by the same symbols, and duplicate description is omitted. Also, the scale of the members in the drawings may differ from the actual scale. In this specification, the expression indicating a numerical range: “from . . . through . . . ” means that the numerical value described after “from” and the numerical value described after “through” are included in that numerical range as a lower limit and an upper limit, unless otherwise specified.

### <Biological Sensor>

A biological sensor according to the present embodiment will be described. The living body refers to, for example, a human body (human) and animals, such as cattle, horses, pigs, chickens, dogs, cats, and the like. The biological sensor according to the present embodiment is suitably used for the living body, especially for a human body. The present embodiment will be described taking, as an example, a case in which the living body is of a human.

The biological sensor according to the present embodiment is an attachment-type biological sensor configured to be attached to a part of a living body (e.g., skin, scalp, forehead, or the like), thereby performing measurement of biological information. In the present embodiment, a description will be given of a case in which the biological sensor is attached to the skin of a human and measures an electric signal (biological signal) indicating biological information of the human.

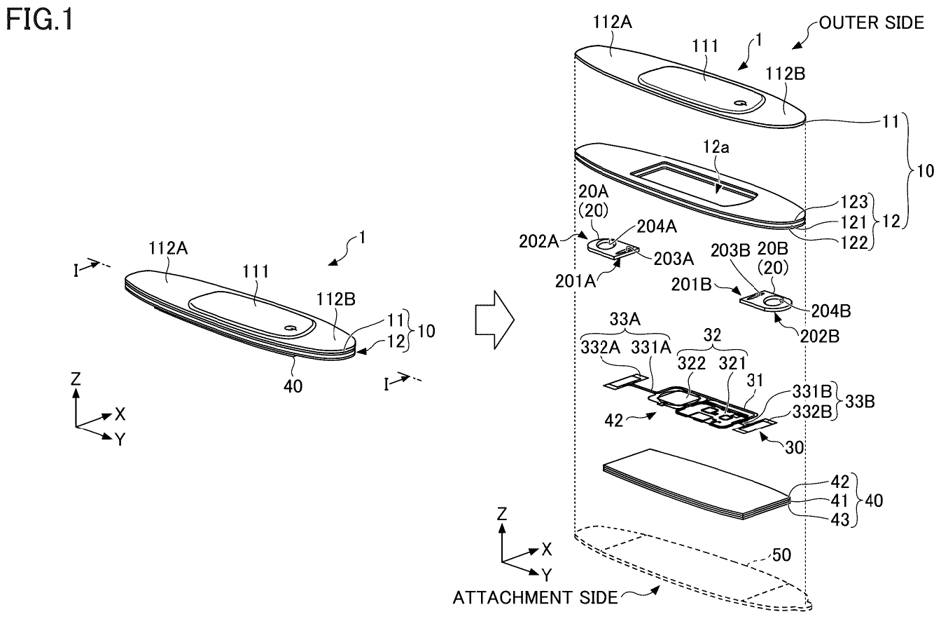

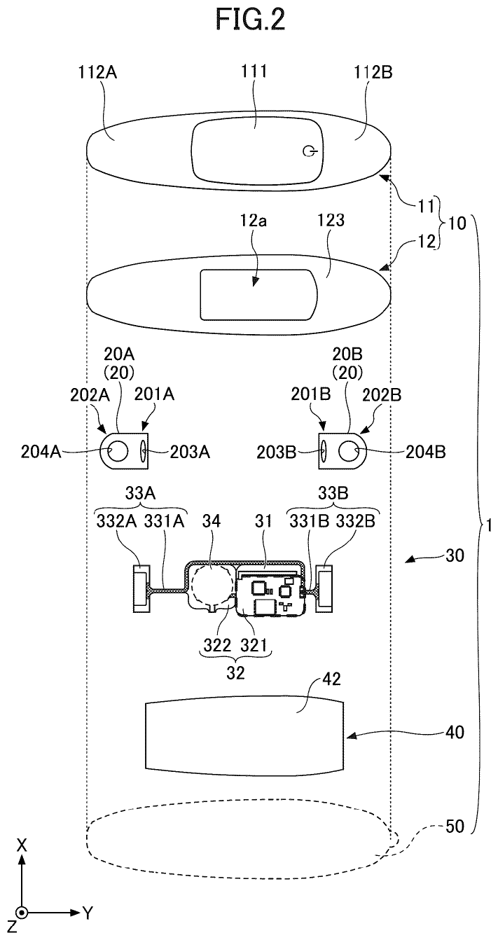

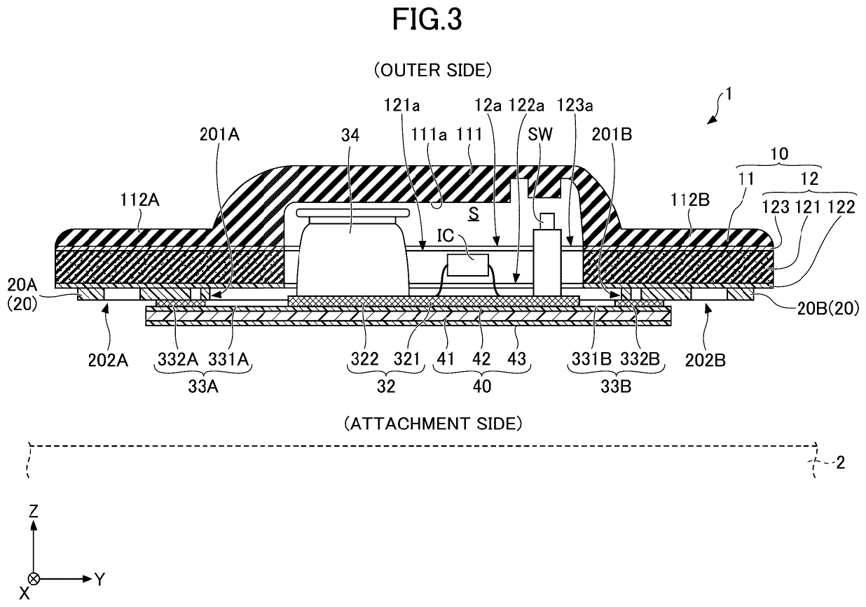

FIG. 1 is a perspective view illustrating the entire configuration of the biological sensor according to the present embodiment. The left-hand view of FIG. 1 illustrates the external appearance of the biological sensor according to the present embodiment, and the right-hand view of FIG. 1 illustrates a state in which the parts of the biological sensor according to the present embodiment are exploded. FIG. 2 is a plan view illustrating examples of the parts of the biological sensor. FIG. 3 is a longitudinal cross-sectional view of the biological sensor taken along the line I-I in FIG. 1.

As illustrated in FIGS. 1 and 2, a biological sensor 1 is a plate-like (sheet-like) member formed in a substantially elliptical shape in a plan view. As illustrated in FIGS. 2 and 3, the biological sensor 1 includes a first layer member 10, an electrode 20, a sensor portion 30, and a second layer member 40, and is formed by stacking the first layer member 10, the electrode 20, and the second layer member 40 in this order from the first layer member 10 side toward the second layer member 40 side. According to the biological sensor 1, the first layer member 10, the electrode 20, and the second layer member 40 form an attachment surface to be attached to a skin 2, which is an example of the living body. The biological sensor 1 attaches the attachment surface to the skin 2 and measures a potential difference (polarization voltage) between the skin 2 and the electrode 20, thereby measuring an electric signal (biological signal) indicating biological information of a subject.

In FIGS. 1 to 3, using a three-dimensional orthogonal coordinate system having three axis directions (X-axis direction, Y-axis direction, and Z-axis direction), the transverse direction of the biological sensor is an X-axis direction, the longitudinal direction of the biological sensor is a Y-axis direction, and the height direction (thickness direction) of the biological sensor is a Z-axis direction. The side (outer side) opposite to the side on which the biological sensor 1 is attached to the living body (subject) (attachment side) is referred to as a +Z-axis direction, and the attachment side is referred to as a −Z-axis direction. In the following description, for the sake of convenience, the +Z-axis direction may be referred to as an upper side or above, and the −Z-axis direction may be referred to as a lower side or below. However, this does not represent a universal vertical relationship.

The biological signal is, for example, an electric signal indicating an electrocardiogram waveform, an electroencephalogram, a pulse, or the like.

In use of the biological sensor 1, the inventors of the present application focused on how the thickness, the area, and the tack force of the electrode 20, which is provided on the living body side, i.e., on the skin 2 side, of the first layer member 10, and the covering percentage of the electrode 20 with respect to the first layer member 10 influence suppression of noise generated during use of the biological sensor 1 and an attachment performance to the living body. The inventors of the present application have found that, by reducing the volume and the adhesiveness of the electrode 20 to be within predetermined ranges and reducing the covering percentage of the electrode 20 with respect to the first layer member 10, the adhesion state of the electrode 20 to the surface of the skin 2 can be maintained and the adhesiveness to the surface of the skin 2 can be enhanced, thereby suppressing generation of noise detected during use of the biological sensor 1 and enhancing an attachment performance of the biological sensor 1 to the living body.

### [First Layer Member]

As illustrated in FIGS. 1 and 2, the first layer member 10 includes a cover member 11 and an upper sheet 12 that are stacked in this order. The cover member 11 and the upper sheet 12 have substantially the same outer shape in a plan view.

### (Cover Member)

As illustrated in FIG. 3, the cover member 11 is positioned on the outermost side (+Z-axis direction) of the biological sensor 1, and is adhered to the upper surface of the upper sheet 12. The cover member 11 includes: a projection 111 that projects in a substantially dome shape in the height direction (+Z-axis direction) in FIG. 1, the projection 111 being in a center region in the longitudinal direction (Y-axis direction); and flat portions 112A and 112B provided at both ends of the cover member 11 in the longitudinal direction (Y-axis direction). The upper and lower surfaces of the projection 111, and the upper and lower surfaces of the flat portions 112A and 112B are formed to be flat.

The cover member 11 has an opening on the inner side (attachment side) of the projection 111 so as to have a recess 111a formed in a recessed shape on the skin 2 side. The recess 111a only needs to have a size sufficient to house at least a part of the sensor portion 30. A housing space S in which the sensor portion 30 is housed is formed, on the inner side (attachment side) of the projection 111, by the recess 111a at the inner surface of the projection 111, the electrode 20, and the second layer member 40.

As a material forming the cover member 11, a flexible material, such as silicone rubber, fluororubber, urethane rubber, or the like, can be used. The cover member 11 may be formed by stacking the flexible material on the surface of a support that is formed of a base resin, such as polyethylene terephthalate (PET) or the like. The cover member 11 formed using the flexible material or the like protects the sensor portion 30 disposed in the housing space S of the cover member 11, and absorbs an impact applied to the biological sensor 1 from the upper surface side to reduce the impact applied to the sensor portion 30.

The thickness of the upper surface and the side walls of the projection 111 may be larger than that of the flat portions 112A and 112B. Thus, the flexibility of the projection 111 can be lower than that of the flat portions 112A and 112B, and the sensor portion 30 can be protected from an external force applied to the biological sensor 1.

The thickness of the upper surface and the side walls of the projection 111 can be appropriately designed and may be, for example, from 1.5 mm through 3 mm. The thickness of the flat portions 112A and 112B can also be appropriately designed and may be, for example, from 0.5 mm through 1 mm.

The flat portions 112A and 112B, which are thinner, have higher flexibility than that of the projection 111. Thus, when the biological sensor 1 is attached to the skin 2, they readily deform in accordance with deformation of the surface of the skin 2 caused by body movements, such as extension, bending, twisting, and the like. This can reduce stress applied to the flat portions 112A and 112B in response to deformation of the surface of the skin 2, and can suppress peeling of the biological sensor 1 off from the skin 2.

The outer peripheral portions of the flat portions 112A and 112B may have a shape in which the thickness gradually decreases toward the respective ends. This can further increase the flexibility of the outer peripheral portions of the flat portions 112A and 112B, and can improve sensation during attachment of the biological sensor 1 to the skin 2 compared to a case in which the thickness of the outer peripheral portions of the flat portions 112A and 112B are not made smaller.

The hardness (strength) of the cover member 11 can be appropriately designed to have a desirable magnitude, and, for example, may be from 40 through 70. When the hardness of the cover member 11 is within the above preferable range, the upper sheet 12, the electrode 20, and the second layer member 40 can readily deform in accordance with the movement of the skin 2 without being influenced by the cover member 11 when the skin 2 is extended by the body movements. The hardness (how hard it is) refers to Shore A hardness. In the present specification, the Shore A hardness refers to a value as measured in accordance with ISO7619 (JIS K 6253:2012). The Shore A hardness is a type A durometer hardness as measured by a rubber hardness meter (type A durometer) using a type A (cylindrical) indenter.

### (Upper Sheet)

As illustrated in FIG. 3, the upper sheet 12 is adhered to the lower surface of the cover member 11. The upper sheet 12 has a through-hole 12a at a position facing the projection 111 of the cover member 11. Owing to the through-hole 12a, a sensor body 32 of the sensor portion 30 can be housed in the housing space S, formed by the recess 111a at the inner surface of the cover member 11 and the through-hole 12a, without being blocked by the upper sheet 12.

The upper sheet 12 includes: a first base 121; a first adhesive layer 122 that is provided at one surface of the first base 121 facing the electrode 20 and to which the electrode 20 is attached; and an upper adhesive layer 123 that is provided at the surface of the first base 121 opposite to the surface facing the electrode 20.

### ((First Base))

As illustrated in FIG. 3, the first base 121 is provided on the attachment side that is the opening side of the cover member 11. The first base 121 is formed in a sheet shape. The first base 121 may be formed of a porous body having a porous structure and having flexibility, waterproofness, and moisture permeability. As the porous body, for example, a foamed material (foamed body) having cells, such as open cells, closed cells, and semi-closed cells, can be used. As such, water vapor derived from sweat or the like generated from the skin 2, to which the biological sensor 1 is attached, can be released to the exterior of the biological sensor 1 through the first base 121.

The moisture permeability of the first base 121 is preferably from 100 g/(m²·day) through 5,000 g/(m²·day). By setting the moisture permeability of the first base 121 to be in the range of from 100 g/(m²·day) through 5,000 g/(m²·day), the water vapor entering the first base 121 from one surface can pass through the first base 121, and can be stably released from the other surface.

As the material forming the first base 121, a thermoplastic resin can be used, and examples of the thermoplastic resin include polyurethane-based resins, polystyrene-based resins, polyolefin-based resins, silicone-based resins, acrylic resins, vinyl chloride-based resins, polyester-based resins, and the like. As the first base 121, for example, FOLEC available from INOAC CORPORATION may be used.

The thickness of the first base 121 may be appropriately set, and, for example, may be from 0.5 mm through 1.5 mm.

The first base 121 has a through-hole 121a at a position facing the projection 111 of the cover member 11. When the first adhesive layer 122 and the upper adhesive layer 123 are provided on the surface of the first base 121 other than the through-hole 121a, through-holes 122a and 123a can also be formed in the first adhesive layer 122 and the upper adhesive layer 123. The through-holes 121a, 122a, and 123a form the through-hole 12a.

The first base 121 may be a base having no porous structure as long as the base has flexibility, waterproofness, and moisture permeability. Because the first base 121 has flexibility, waterproofness, and moisture permeability, the first base 121 can be readily stretched in the state of contacting the skin 2. Thus, the state of contacting the skin 2 can be maintained, and also the entry of liquid into the gap between the first base 121 and the upper adhesive layer 123 can be suppressed. Further, water vapor derived from sweat or the like generated from the skin 2, to which the biological sensor 1 is attached, can be released to the exterior of the biological sensor 1 through the first base 121. Therefore, the upper sheet 12 readily maintains adhesion durability.

As the material of the base material having no porous structure, a thermoplastic resin can be used similar to the above, and examples of the thermoplastic resin include polyurethane-based resins, polystyrene-based resins, polyolefin-based resins, silicone-based resins, acrylic resins, vinyl chloride-based resins, polyester-based resins, and the like. When the first base 121 is formed of a base material having no porous structure, a polyurethane sheet, such as, for example, ESMER URS available from Nihon Matai Co., Ltd., can be used as the first base 121.

((First Adhesive layer))

As illustrated in FIG. 3, the first adhesive layer 122 is attached to one surface of the first base 121 facing the electrode 20. The first adhesive layer 122 is positioned at a surface of the first base 121 facing the living body (−Z-axis direction), and has the function of adhering the skin 2 and the first base 121 to each other, the function of adhering the first base 121 and a second base 41 to each other, and the function of adhering the first base 121 and the electrode 20 to each other.

The first adhesive layer 122 may have moisture permeability. As such, as described below, water vapor derived from sweat or the like generated from the skin 2, to which the biological sensor 1 is attached, can be escaped to the first base 121 through the first adhesive layer 122, and can be released to the exterior of the biological sensor 1 through the first base 121. When the first base 121 has a cell structure as described above, water vapor can be released to the exterior of the biological sensor 1 through the first adhesive layer 122. This can prevent sweat or water vapor from accumulating at the interface between the skin 2, on which the biological sensor 1 is attached, and the first layer member 10. As a result, it is possible to prevent the adhesive strength of the first adhesive layer 122 from weakening due to the moisture accumulated at the interface between the skin 2 and the first adhesive layer 122, and prevent peeling of the biological sensor 1 off from the skin 2.

Preferably, the moisture permeability of the first adhesive layer 122 is, for example, 1 g/(m²·day) or more. The moisture permeability of the first adhesive layer 122 may be 10,000 g/(m²·day) or less. As long as the moisture permeability of the first adhesive layer 122 is 1 g/(m²·day) or more, when the first adhesive layer 122 is attached to the skin 2, sweat or the like delivered from the first adhesive layer 122 can be released toward the exterior. This can reduce the burden on the skin 2.

The material forming the first adhesive layer 122 is preferably a material having pressure-sensitive adhesiveness. For example, an acrylic pressure-sensitive adhesive may be used.

The first adhesive layer 122 may be adhesive tape formed of the above material.

A wavy pattern (web pattern) may be formed on the surface of the first adhesive layer 122. This wavy pattern is formed by repeatedly and alternatingly arranging recesses for a thickness smaller than that of the other portions (or for zero thickness). As the first adhesive layer 122, for example, adhesive tape having a web pattern formed on a surface of the adhesive tape may be used. The first adhesive layer 122 has a web pattern on the surface, and as a result, the surface of the first adhesive layer 122 includes both of: portions in which an adhesive is likely to contact the living body; and portions in which the adhesive is unlikely to contact the living body. Because the surface of the first adhesive layer 122 includes both of the portions in which the adhesive is present and the portions in which the adhesive is absent, the portions that are likely to contact the living body can be sparsely located on the surface of the first adhesive layer 122. The moisture permeability of the first adhesive layer 122 tends to increase as the adhesive is thinner. Therefore, by forming the web pattern on the surface of the first adhesive layer 122 and providing the surface of the first adhesive layer 122 with portions in which the adhesive is thinner, it is possible to enhance the moisture permeability while maintaining the adhesive strength, compared to a case in which the web pattern is not formed. The shape of the recess may be a straight shape or a circular shape, in addition to a wavy shape.

The widths of an adhesive-applied portion and an adhesive-free portion may be appropriately designed. For example, the width of the adhesive-applied portion is preferably from 500 μm through 1,000 μm, and the width of the adhesive-free portion is preferably from 1,500 μm through 5,000 μm. When the widths of the adhesive-applied portion and the adhesive-free portion are within the above preferable ranges, the first adhesive layer 122 can exhibit excellent moisture permeability while maintaining the adhesive strength.

The thickness of the first adhesive layer 122 may be desirably set, and is preferably from 10 μm through 300 μm, more preferably from 50 μm through 200 μm, and further preferably from 70 μm through 110 μm. When the thickness of the first adhesive layer 122 is from 10 μm through 300 μm, the biological sensor 1 can be reduced in thickness.

The adhesive strength of the first adhesive layer 122 may be desirably set, and, for example, is preferably from 3.0 N/10 mm through 20 N/10 mm, more preferably from 4.0 N/10 mm through 15 N/10 mm, and further preferably from 5.0 N/10 mm through 10 N/10 mm, with respect to a Bakelite board. When the adhesive strength of the first adhesive layer 122 is from 3.0 N/10 mm through 20 N/10 mm, an attachment performance of the biological sensor 1 to the living body can be enhanced because the first adhesive layer 122 forms a part of the attachment surface of the biological sensor 1 to the surface of the skin 2.

### ((Upper Adhesive Layer))

As illustrated in FIG. 3, the upper adhesive layer 123 is attached to the surface of the first base 121 opposite to the surface facing the electrode 20. The upper adhesive layer 123 is attached to the upper surface of the first base 121 and at a position corresponding to the flat surface on the attachment side (−Z-axis direction) of the cover member 11. The upper adhesive layer 123 has the function of adhering the first base 121 and the cover member 11 to each other.

As the material forming the upper adhesive layer 123, a silicone-based adhesive, silicone tape, or the like, can be used.

The thickness of the upper adhesive layer 123 may be appropriately set, and, for example, may be from 10 μm through 300 μm.

### [Electrode]

As illustrated in FIG. 3, the electrode 20 is attached to the lower surface of the first adhesive layer 122 on the attachment side (−z-axis direction) in a state in which a part of the electrode 20 on the sensor body 32 side is connected to interconnects 331A and 331B and is held between the first adhesive layer 122 and a lower adhesive layer 42. The electrode 20 contacts the living body at a portion that is not held between the first adhesive layer 122 and the lower adhesive layer 42. When the biological sensor 1 is attached to the skin 2, the electrode 20 contacts the skin 2, thereby enabling detecting biological signals. The electrode 20 may be embedded in the second base 41 in a state in which the electrode 20 is exposed so as to be able to contact the skin 2.

The electrode 20 is formed by a pair of electrodes 20A and 20B. As illustrated in FIG. 3, the electrode 20A is disposed on the left-hand side in the drawing, and the electrode 20B is disposed on the right-hand side in the drawing. One end (inner side) of the electrode 20A in the longitudinal direction (Y-axis direction) contacts a terminal 332A, and one end (inner side) of the electrode 20B in the longitudinal direction (Y-axis direction) contacts a terminal 332B. The pair of electrodes 20A and 20B have substantially the same shape.

The one end of the electrode 20A that contacts the terminal 332A of the sensor portion 30 is referred to as a facing portion 201A, and the one end of the electrode 20B that contacts the terminal 332B of the sensor portion 30 is referred to as a facing portion 201B. A portion of the electrode 20A that does not contact the terminal 332A (the other end (outer side) in the longitudinal direction (Y-axis direction)) is referred to as an exposed portion 202A, and a portion of the electrode 20B that does not contact the terminal 332B (the other end (outer side) in the longitudinal direction (Y-axis direction)) is referred to as an exposed portion 202B.

The electrode 20 may have any shape, such as a sheet shape or the like.

No particular limitation is imposed on the shape of the electrode 20 in a plan view. The electrode 20 may be designed to have a shape that is appropriate in accordance with applications or the like. As illustrated in FIG. 2, the electrode 20A or 20B may be formed such that in a plan view, the one end, i.e., the facing portion 201A or 201B, is formed in an arc shape, and the other end, i.e., the exposed portion 202A or 202B, is formed in a rectangular shape.

As illustrated in FIGS. 2 and 3, the electrode 20A or 20B is provided at the one end (inner side) in the longitudinal direction (Y-axis direction), and may have: a through-hole 203A or 203B formed at the one end (inner side) and having an oval shape that is thin and long in the width direction (X-axis direction); and a through-hole 204A or 204B formed to be circular at the other end (outer side) in the longitudinal direction (Y-axis direction). Thus, the electrode 20 can expose the first adhesive layer 122 from the through-holes 203A and 203B and the through-holes 204A and 204B to the attachment side in a state of being attached to the first adhesive layer 122, thereby enhancing adhesiveness between the electrode 20 and the skin 2. No particular limitation is imposed on the number of the through-holes 203A and 203B or the through-holes 204A and 204B. The number of the through-holes 203A and 203B or the through-holes 204A and 204B may be appropriately set in accordance with the sizes and the like of the facing portions 201A and 201B of the electrode 20.

The electrode 20 is preferably an electrode having adhesiveness (adhesive electrode). When the electrode 20 is an adhesive electrode, the electrode 20 can be formed by an adhesive electrode sheet, i.e., a sheet formed of an adhesive conductive composition containing a conductive polymer, a binder resin, and a moisturizer.

As the conductive polymer, for example, it is possible to use a polythiophene-based conductive polymer, a polyaniline-based conductive polymer, a polypyrrole-based conductive polymer, a polyacetylene-based conductive polymer, a polyphenylene-based conductive polymer, a derivative of the above-listed polymers, a composite of the above-listed polymers, or the like. These may be used alone or in combination. Of these, it is preferable to use composites in which polythiophene is doped with polyaniline as a dopant. Of the composites of polythiophene and polyaniline, it is more preferable to use PEDOT/PSS in which poly 3,4-ethylenedioxythiophene (PEDOT) is doped with polystyrene sulfonic acid (poly 4-styrenesulfonate; PSS) because of low contact impedance with the living body and high conductivity.

The binder resin is formed of an aqueous emulsion adhesive. The aqueous emulsion adhesive has the function of enhancing adhesiveness and flexibility of the electrode 20. Therefore, when the aqueous emulsion adhesive is contained in the electrode 20, the electrode 20 can be made low in elasticity, and can be increased in followability to irregularities on the surface of the skin 2.

As the aqueous emulsion adhesive, an acrylic emulsion adhesive can be used.

The acrylic emulsion adhesive preferably uses a silane-based emulsion adhesive containing a water-dispersible copolymer and an organic liquid component compatible with the water-dispersible copolymer.

The water-dispersible copolymer is a polymer obtained by copolymerizing a silane-based monomer copolymerizable with a (meth)acrylic acid alkyl ester, with a monomer mixture containing a (meth)acrylic acid alkyl ester.

The monomer mixture containing a (meth)acrylic acid alkyl ester is a monomer mixture containing a (meth)acrylic acid alkyl ester as a main component, and preferably containing a (meth)acrylic acid alkyl ester by from 50 wt % through 100 wt %.

As the (meth)acrylic acid alkyl ester, a linear or branched alkyl ester containing an alkyl group having from 1 through 15 carbon atoms, and preferably from 1 through 9 carbon atoms is used. Specific examples include (meth)acrylic acid alkyl esters containing linear or branched alkyl groups, such as methyl (meth)acrylate, ethyl (meth)acrylate, n-butyl (meth)acrylate, isobutyl (meth)acrylate, pentyl (meth)acrylate, hexyl (meth)acrylate, heptyl (meth)acrylate, n-octyl (meth)acrylate, isooctyl (meth)acrylate, 2-ethylhexyl (meth)acrylate, n-nonyl (meth)acrylate, isononyl (meth)acrylate, decyl (meth)acrylate, undecyl (meth)acrylate, tridecyl (meth)acrylate, and the like. These may be used alone or in combination.

The monomer mixture containing the (meth)acrylic acid alkyl ester may contain a carboxyl group-containing monomer copolymerizable with the (meth)acrylic acid alkyl ester.

No particular limitation is imposed on the carboxyl group-containing monomer copolymerizable with the (meth)acrylic acid alkyl ester as long as the carboxyl group-containing monomer is a polymerizable compound containing a carboxyl group in the structure and is copolymerizable with the (meth)acrylic acid alkyl ester. Examples of the carboxyl group-containing monomer include (meth)acrylic acid, itaconic acid, maleic acid, maleic anhydride, 2-methacryloyloxyethylsuccinic acid, and the like. Acrylic acid is especially preferable.

From the viewpoint of hydrolysis of the silane-based monomer and adjustment of obtained adhesiveness, the carboxyl group-containing monomer is preferably contained by from 0.1 wt % through 10 wt % per 100 wt % of the monomer mixture containing the (meth)acrylic acid alkyl ester.

No particular limitation is imposed on the silane-based monomer copolymerizable with the (meth)acrylic acid alkyl ester as long as the silane-based monomer is a polymerizable compound containing a silicon atom and is copolymerizable with the (meth)acrylic acid alkyl ester. From the viewpoint of high copolymerizability with the (meth)acrylic acid alkyl ester, a silane compound having a (meth)acryloyl group, such as a (meth)acryloyloxyalkylsilane derivative or the like, is preferable. Examples of the silane-based monomer include 3-(meth)acryloyloxypropyltrimethoxysilane, 3-(meth)acryloyloxypropyltriethoxysilane, 3-(meth)acryloyloxypropylmethyldimethoxysilane, 3-(meth)acryloyloxypropylmethyldiethoxysilane, and the like. These silane-based monomers may be used alone or in combination.

As the silane-based monomer other than the above silane-based monomers, for example, it is possible to use vinyltrimethoxysilane, vinyltriethoxysilane, 4-vinylbutyltrimethoxysilane, 4-vinylbutyltriethoxysilane, 8-vinyloctyltrimethoxysilane, 8-vinyloctyltriethoxysilane, 10-methacryloyloxydecyltrimethoxysilane, 10-acryloyloxydecyltrimethoxysilane, 10-methacryloyloxydecyltriethoxysilane, 10-acryloyloxydecyltriethoxysilane, and the like.

The silane-based monomer is preferably copolymerized with the monomer mixture containing the (meth)acrylic acid alkyl ester by from 0.005 wt % through 2 wt % per 100 wt % of the monomer mixture containing the (meth)acrylic acid alkyl ester.

By copolymerizing the silane-based monomer with the monomer mixture containing the (meth)acrylic acid alkyl ester, the silane compound serving as a crosslinking point can be uniformly present in the molecule of the obtained copolymer. As such, the aqueous emulsion adhesive is water dispersible, but is excellent in aggregation force because the interior and exterior of the particles of the aqueous emulsion adhesive are uniformly crosslinked. Thus, the aqueous emulsion adhesive is low in skin irritation by the addition of the organic liquid component, and also has excellent fixability and excellent sweat-resistant fixability.

If necessary, the water-dispersible copolymer may be a copolymer obtained through copolymerization of a monomer copolymerizable with a (meth)acrylic acid alkyl ester other than the above silane-based monomer and carboxyl group-containing monomer. The monomer copolymerizable with the (meth)acrylic acid alkyl ester other than the silane-based monomer and carboxyl group-containing monomer can be used, for example, in order to adjust the aggregation force of the electrode 20 when forming the aqueous emulsion adhesive in a sheet-like form or the like, or in order to increase compatibility with the organic liquid component. The amount of the monomer copolymerizable with the (meth)acrylic acid alkyl ester other than the silane-based monomer and carboxyl group-containing monomer can be desirably set in accordance with the intended purpose in place of a part of the content of the (meth)acrylic acid alkyl ester.

Examples of the monomer copolymerizable with the (meth)acrylic acid alkyl ester other than the silane-based monomer and carboxyl group-containing monomer include, for example: sulfoxyl group-containing monomers, such as styrene sulfonic acid, allyl sulfonic acid, sulfopropyl (meth)acrylate, (meth)acryloyloxynaphthalene sulfonic acid, acrylamidomethylpropane sulfonic acid, and the like; hydroxyl group-containing monomers, such as hydroxyethyl (meth)acrylate, hydroxypropyl (meth)acrylate, and the like; amide group-containing monomers, such as (meth)acrylamide, dimethyl (meth)acrylamide, N-butylacrylamide, N-methylol (meth)acrylamide, N-methylolpropane (meth)acrylamide, and the like; alkylaminoalkyl (meth)acrylates, such as aminoethyl (meth)acrylate, dimethylaminoethyl (meth)acrylate, tert-butylaminoethyl (meth)acrylate, and the like; alkoxyalkyl (meth)acrylates, such as methoxyethyl (meth)acrylate, ethoxyethyl (meth)acrylate, and the like; alkoxy group-(or side-chain-ether group)-containing (meth)acrylates, such as methoxyethylene glycol (meth)acrylate, tetrahydrofurfuryl (meth)acrylate, methoxyethylene glycol (meth)acrylate, methoxydiethylene glycol (meth)acrylate, methoxypolyethylene glycol (meth)acrylate, methoxypolypropylene glycol (meth)acrylate, and the like; and vinyl-based monomers, such as methacrylonitrile, vinyl acetate, vinyl propionate, N-vinyl-2-pyrrolidone, methylvinylpyrrolidone, vinylpyridine, vinylpiperidine, vinylpyrimidine, vinylpiperazine, vinylpyrazine, vinylpyrrole, vinylimidazole, vinylcaprolactam, vinyloxazole, vinylmorpholine, and the like. These may be used alone or in combination.

The water-dispersible polymer can be prepared as an aqueous dispersion liquid of a (meth)acrylic acid alkyl ester copolymer by subjecting a mixture of a monomer mixture containing a (meth)acrylic acid alkyl ester and a silane-based monomer, to typical emulsion polymerization.

As the polymerization method, typical batch polymerization, continuous dropping polymerization, divided dropping polymerization, or the like can be employed. The polymerization temperature is, for example, from 20° C. through 100° C.

No particular limitation is imposed on the polymerization initiator used for the polymerization. Typical components used as a polymerization initiator can be used.

A chain transfer agent may be used for the polymerization in order to adjust the degree of polymerization. No particular limitation is imposed on the chain transfer agent. Typical components used as a chain transfer agent can be used.

In addition to the above methods, the water-dispersible copolymer may be prepared by obtaining a copolymer of a silane-based monomer and a monomer mixture containing a (meth)acrylic acid ester by a method other than emulsion polymerization, and then dispersing the copolymer in water with an emulsifier.

When the organic liquid component contained in the acrylic emulsion adhesive is included in the water-dispersible copolymer, it is possible to maintain favorable adhesiveness to the surface of the skin 2, reduce damage to keratin during peeling, and also reduce pain during peeling.

The organic liquid component is preferably liquid at room temperature and has favorable compatibility with the water-dispersible copolymer. The “compatibility” means that the organic liquid component is uniformly dissolved and included in the water-dispersible copolymer, and that no separation is visually confirmed.

Examples of the organic liquid component include, for example: esters between monobasic acid or polybasic acid having from 8 through 18 carbon atoms and branched alcohol having from 14 through 18 carbon atoms; and esters between unsaturated fatty acid or branched acid having from 14 through 18 carbon atoms and tetravalent or lower alcohol.

Examples of the esters between monobasic acid or polybasic acid having from 8 through 18 carbon atoms and branched alcohol having from 14 through 18 carbon atoms include isostearyl laurate, isocetyl myristate, octyldodecyl myristate, isostearyl palmitate, isocetyl stearate, octyldodecyl oleate, diisostearyl adipate, diisocetyl sebacate, trioleyl trimellitate, triisocetyl trimellitate, and the like.

Examples of the unsaturated fatty acid or branched acid having from 14 through 18 carbon atoms include myristoleic acid, oleic acid, linoleic acid, linolenic acid, isopalmitic acid, isostearic acid, and the like.

Examples of the tetravalent or lower alcohol include ethylene glycol, propylene glycol, glycerin, trimethylolpropane, pentaerythritol, sorbitan, and the like.

The content of the organic liquid component can be appropriately set in accordance with, for example, types of the water-dispersible copolymer and the organic liquid component, and is, for example, from 20 wt % through 80 wt % per 100 wt % of the water-dispersible copolymer.

When the acrylic emulsion adhesive is a silane-based emulsion adhesive, specifically, a silane-based emulsion adhesive containing 2-ethylhexyl acrylate, methyl methacrylate, acrylic acid, and 3-methacryloxypropyltrimethoxysilane can be used as the acrylic emulsion adhesive.

Two- or three-component acrylic emulsion adhesives containing a carboxyl group-containing monomer and a monomer mixture containing a (meth)acrylic acid alkyl ester can be used as the acrylic emulsion adhesive. These may contain a solvent or any other component in appropriate predetermined amounts within a range in which the solvent or any other component can exhibit their performance.

The monomer mixture containing the (meth)acrylic acid alkyl ester contained in the two- or three-component acrylic emulsion adhesive is similar to the monomer mixture containing the (meth)acrylic acid alkyl ester contained in the above silane-based emulsion adhesive, and details of the monomer mixture will be omitted.

The carboxyl group-containing monomer is preferably a carboxyl group-containing monomer copolymerizable with the (meth)acrylic acid alkyl ester. The carboxyl group-containing monomer copolymerizable with the (meth)acrylic acid alkyl ester is similar to the carboxyl group-containing monomer contained in the monomer mixture containing the (meth)acrylic acid alkyl ester, and details of the carboxyl group-containing monomer will be omitted.

As the two-component acrylic emulsion adhesive, specifically, an adhesive containing: 2-ethylhexyl acrylate, i.e., a monomer mixture containing a (meth)acrylic acid alkyl ester; and acrylic acid, i.e., a carboxyl group-containing monomer mixture, can be used.

As the three-component acrylic emulsion adhesive, specifically, an adhesive containing: 2-ethylhexyl acrylate and methyl methacrylate, i.e., a monomer mixture containing a (meth)acrylic acid alkyl ester; and acrylic acid, i.e., a carboxyl group-containing monomer mixture, can be used.

The average particle diameter of the aqueous emulsion adhesive is preferably from 100 nm through 1.0 μm, more preferably from 100 nm through 500 nm, and further preferably from 100 nm through 300 nm. When the average particle diameter is within the above preferable range, the electrode 20 can be provided with adhesive strength and waterproofness.

No particular limitation is imposed on the shape of the aqueous emulsion adhesive. The shape of the aqueous emulsion adhesive may be, for example, a spherical shape, an ellipsoidal shape, a spindle shape, a crushed shape, a plate shape, a column shape, or the like.

The average particle diameter refers to a volume average particle diameter in accordance with the effective diameter. The average particle diameter is a particle diameter (median diameter) in a particle size distribution curve obtained by measuring the particle size distribution of an emulsion adhesive or an acrylic emulsion adhesive through, for example, laser diffraction and scattering, dynamic light scattering, or the like, and the particle diameter is a particle diameter when the cumulative amount of particles counted from the particles having smaller particle diameters accounts for 50% on a volume basis.

The content of the binder resin is preferably from 35 wt % through 90 wt %, more preferably from 40 wt % through 85 wt %, and further preferably from 50 wt % through 80 wt %. When the content of the binder resin is within the above preferable range, it is possible to impart adhesive strength and flexibility to the electrode 20, and suppress a reduction in conductivity.

The moisturizer has the functions of increasing conductivity of the electrode 20 and increasing the degree of adhesive strength and flexibility. Examples of the moisturizer include, for example: polyol compounds, such as glycerin, ethylene glycol, propylene glycol, sorbitol, polymers of these, and the like; and aprotic compounds, such as N-methylpyrrolidone (NMP), dimethyl formaldehyde (DMF), N—N′-dimethylacetamide (DMAc), dimethyl sulfoxide (DMSO), and the like. These may be used alone or in combination. Of these, glycerin is preferable from the viewpoint of compatibility with other components.

The content of the moisturizer is preferably from 2 wt % through 60 wt %, more preferably from 3 wt % through 50 wt %, and further preferably from 5 wt % through 35 wt %, per 100 wt % of the electrode. When the content of the moisturizer is within the above preferable range, it is possible to enhance the adhesive strength of the electrode 20, maintain high adhesiveness to the surface of the skin 2, decrease the storage modulus, and increase the viscoelasticity. Thus, the magnitude of noise generated during use can be lowered. Also, it is possible to suppress absorption of external water by the electrode 20, thereby suppressing swelling.

The thickness of the electrode 20 is 15 μm or more, preferably from 20 μm through 100 μm, more preferably from 25 μm through 90 μm, and further preferably from 30 μm through 80 μm. When the thickness of the electrode 20 is 15 μm or more, the electrode 20 can be provided with sufficient strength, flexibility, low resistance, and conductive stability during deformation.

The thickness of the electrode 20 is a length of the electrode 20 in a direction perpendicular to the surface of the electrode 20. The thickness of the electrode 20 is, for example, a thickness as measured at a given site in a cross section of the electrode 20. When measurement is performed at a plurality of given sites, the average value of the thicknesses measured at the plurality of given sites may be used as the thickness of the electrode 20.

The area of the electrode 20 is from 2.0 cm² through 5.0 cm², preferably from 2.5 cm² through 4.5 cm², and more preferably from 2.7 cm² through 4.0 cm². When the area of the electrode 20 is from 2.0 cm² through 5.0 cm², the electrode 20 can have sufficient conductive stability and adhesive strength.

The area of the electrode 20 can be measured by a typical measurement method that is, for example, as follows. Specifically, using typical image analysis software (e.g., ImageJ or the like), the surface of the electrode 20 is photographed, the obtained image is binarized, and the area of the electrode 20 expressed in a unit of dot (pixel) is calculated.

The tack force of the electrode 20 is 60 gf/Φ5 mm or more, preferably 63 gf/Φ5 mm or more, and more preferably 65 gf/Φ5 mm or more. No particular limitation is imposed on the upper limit of the tack force of the electrode 20. However, the upper limit of the tack force of the electrode 20 can be appropriately selected in accordance with the size, the shape, the material, and the like, of the electrode 20, and, for example, may be 200 gf/Φ5 mm or less. When the area of the electrode 20 is 60 gf/Φ5 mm or more, the electrode 20 can have conductivity while maintaining a sufficient adhesive strength.

In the present specification, the tack force is a tack force measured when the electrode 20 is a circle having a diameter of 5 mm. The tack force of the electrode 20 can be measured using a typical measurement method, for example, using a typical tacking tester or the like. As measurement conditions, for example, the load pressed on the electrode 20 may be 50 gf, the pressing speed may be 0.01 mm/s, the retention time may be 1.0 s, and the pulling speed may be 1 mm/s.

In the bottom view, the covering percentage of the electrode 20 with respect to the first layer member 10 is from 40% through 90%, preferably from 45% through 80%, and further preferably from 50% through 70%. When the covering percentage is from 40% through 90%, it is possible to ensure conductivity while maintaining sufficient strength, flexibility, and adhesiveness of the electrode 20.

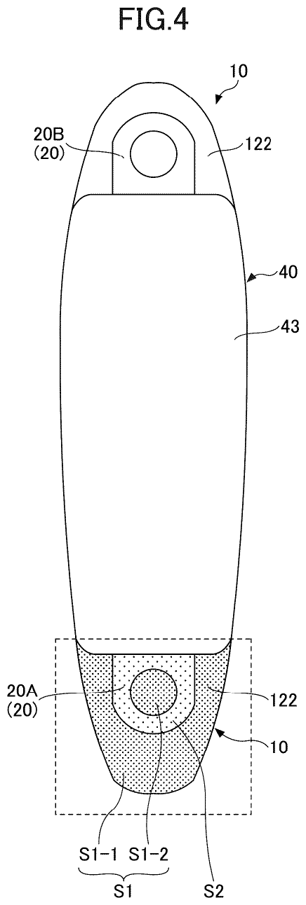

The covering percentage of the electrode 20 with respect to the first layer member 10 is, in the bottom view of the first layer member 10, a percentage of the electrode 20 in a region (area) in which the first layer member 10 and the electrode 20 are exposed as the attachment surface to the living body in the bottom view of the biological sensor, as illustrated in FIG. 4. That is, the covering percentage of the electrode 20 with respect to the first layer member 10 is a ratio of an area S2 of the electrode 20 to the sum of an area S1 of the first layer member 10 and the area S2 of the electrode 20, as shown in the following formula (1).

$$Covering percentage ( \% ) of the \text{electrode} 20 with respect to the first layer member 10 = the area S 2 of the electrode 20 / ( the area S 1 of the first layer member 10 + the area S 2 of the electrode 20 ) \times 100 \qquad ( 1 )$$

### (Sensor Portion)

As illustrated in FIG. 2, the sensor portion 30 has a flexible substrate 31, a sensor body 32, and connection portions 33A and 33B connected to the sensor body 32.

The flexible substrate 31 is a resin substrate on which various parts configured to obtain biological information are mounted. The sensor body 32 and the connection portions 33A and 33B are disposed on the flexible substrate 31.

As illustrated in FIG. 2, the sensor body 32 includes a part-mounting portion 321, serving as a controller, and a battery-mounting portion 322, and obtains biological information.

The part-mounting portion 321 includes various parts mounted on the flexible substrate 31, and obtains biological information. These parts are: a CPU and an integrated circuit configured to process biological signals obtained from the living body and generate biological signal data; a switch SW configured to start-up the biological sensor 1; a flash memory configured to store biological signals; a light-emitting element; and the like. An example of a circuit formed of these parts is omitted. The part-mounting portion 321 is driven by power supplied from a battery 34 mounted on the battery-mounting portion 322.

The part-mounting portion 321 is configured to perform wired or wireless transmission to external devices, such as a drive identifier configured to confirm initial driving, a reader configured to read biological information from the biological sensor 1, and the like.

The battery-mounting portion 322 is disposed between the connection portion 33A and the part-mounting portion 321, and is configured to supply power to an integrated circuit or the like mounted on the part-mounting portion 321. As illustrated in FIG. 2, the battery 34 is mounted on the battery-mounting portion 322.

In the longitudinal direction (Y-axis direction) of the sensor body 32, the connection portions 33A and 33B include: the interconnects 331A and 331B connected to the sensor body 32; and the terminals 332A and 332B provided at the distal ends of the interconnects 331A and 331B and connected to the electrode 20.

As illustrated in FIG. 3, one end of the interconnect 331A or 331B is connected to the electrode 20. As illustrated in FIG. 3, the other end of the interconnect 331A is connected to the switch SW or the like mounted on the part-mounting portion 321 along the outer periphery of the sensor body 32. The other end of the interconnect 331B is connected to the switch SW and the like mounted on the part-mounting portion 321.

The terminal 332A or 332B is disposed in a state in which one end of the terminal 332A or 332B is connected to the interconnect 331A or 331B, and the upper surface of the other end of the terminal 332A or 332B is in contact with the electrode 20 and is held between the first layer member 10 and the second layer member 40.

A publicly known battery can be used as the battery 34. For example, a coin-type battery, such as CR2025 or the like, can be used as the battery 34.

### [Second Layer Member]

As illustrated in FIG. 3, the second layer member 40 is provided on an attachment surface side of the electrode 20 and the sensor portion 30. The second layer member 40 is a support substrate on which the sensor portion 30 is provided, and also forms a part of the attachment surface to the skin 2. As illustrated in FIGS. 1 and 2, the outer shape of the second layer member 40 at both sides in the width direction (X-axis direction) may be formed into substantially the same shape as the outer shape of the first layer member 10 at both sides in the width direction (X-axis direction). The length (Y-axis direction) of the second layer member 40 is formed to be shorter than the length (Y-axis direction) of the cover member 11 and the upper sheet 12. As illustrated in FIG. 3, both of the longitudinal ends of the second layer member 40 are located at positions that hold the interconnects 331A and 331B of the sensor portion 30 between the second layer member 40 and the upper sheet 12, and that overlap with a part of the electrode 20.

The second layer member 40 includes the second base 41, the lower adhesive layer 42 provided on the upper surface of the second base 41, and a second adhesive layer 43 provided on the lower surface of the second base 41. The second base 41, the lower adhesive layer 42, and the second adhesive layer 43 may be formed in the same shape in a plan view. The attachment surface to the skin 2 is formed by the second adhesive layer 43 of the second layer member 40 and the electrode 20. In accordance with the area of the electrode 20 and the second adhesive layer 43, the waterproofness and the moisture permeability are different from position to position on the attachment surface, and thus the adhesiveness can be made different. Therefore, it is possible to enable the waterproofness, the moisture permeability, and the adhesiveness to differ in accordance with the area of the attachment surface of the second adhesive layer 43.

### (Second Base)

The second base 41 can be formed of a flexible resin having appropriate stretchability, flexibility, and toughness. As a material forming the second base 41, for example, it is possible to use a thermoplastic resin including: a polyester-based resin, such as polyethylene terephthalate (PET), polybutylene terephthalate, polytrimethylene terephthalate, polyethylene naphthalate, polybutylene naphthalate, or the like; an acrylic resin, such as polyacrylic acid, polymethacrylic acid, polymethyl acrylate, polymethyl methacrylate (PMMA), polyethyl methacrylate, polybutyl acrylate, or the like; a polyolefin-based resin, such as polyethylene, polypropylene, or the like; a polystyrene-based resin, such as polystyrene, an imide-modified polystyrene, an acrylonitrile-butadiene-styrene (ABS) resin, an imide-modified ABS resin, a styrene-acrylonitrile copolymer (SAN) resin, an acrylonitrile ethylene-propylene-diene styrene (AES) resin, or the like; a polyimide-based resin; a polyurethane-based resin; a silicone-based resin; a polyvinyl chloride-based resin, such as polyvinyl chloride, a vinyl chloride-vinyl acetate copolymer resin, or the like. Of these, a polyolefin-based resin and PET are preferably used. These thermoplastic resins have waterproofness that does not permit permeation of water and water vapor (low in water permeability). Therefore, when the second base 41 is formed of any of these thermoplastic resins, it is possible to suppress the entry of sweat or water vapor generated from the skin 2 into the flexible substrate 31 of the sensor portion 30 through the second base 41 in a state in which the biological sensor 1 is attached to the skin 2 of the living body.

The second base 41 is preferably formed in a flat-plate shape because the sensor portion 30 is disposed on the upper surface via the lower adhesive layer 42.

The thickness of the second base 41 can be appropriately selected and, for example, may be from 1 μm through 300 μm.

### (Lower Adhesive Layer)

As illustrated in FIG. 3, the lower adhesive layer 42 is provided on the upper surface of the second base 41 on the cover member 11 side (+Z-axis direction), and the sensor portion 30 is adhered to the lower adhesive layer 42. Both longitudinal ends of the lower adhesive layer 42 of the second layer member 40 are provided at positions that face the facing portions 201A and 201B of the electrode 20. As such, the facing portions 201A and 201B of the electrode 20 and the terminals 332A and 332B can be held between the upper sheet 12 and the second layer member 40 in a state of being compressed, and the electrode 20 and the terminals 332A and 332B can be electrically connected. The lower adhesive layer 42 can be formed of a material similar to that of the second adhesive layer 43 described below, and details will be omitted. The lower adhesive layer 42 does not necessarily need to be provided, and may be absent.

### (Second Adhesive Layer)

As illustrated in FIG. 3, the second adhesive layer 43 is provided on the lower surface of the second base 41 on the attachment side (−Z-axis direction) and contacts the living body.

The second adhesive layer 43 preferably has pressure-sensitive adhesiveness. By virtue of the pressure-sensitive adhesiveness of the second adhesive layer 43, the biological sensor 1 can be readily attached to the skin 2 by pressing the biological sensor 1 against the skin 2 of the living body.

No particular limitation is imposed on the material of the second adhesive layer 43 as long as the material has pressure-sensitive adhesiveness, and the material is a biocompatible material or the like. Examples of the material forming the second adhesive layer 43 include acrylic pressure-sensitive adhesives, silicone-based pressure-sensitive adhesives, and the like. Silicone-based pressure-sensitive adhesives are preferable.

The acrylic pressure-sensitive adhesive preferably contains an acrylic polymer as a main component. The acrylic polymer can function as a pressure-sensitive adhesive component. The acrylic polymer for use is a polymer obtained through polymerization of a monomer component containing a (meth)acrylic acid ester, such as isononyl acrylate, methoxyethyl acrylate, or the like, as a main component and a monomer copolymerizable with a (meth)acrylic acid ester, such as acrylic acid or the like, as an optional component.

The acrylic pressure-sensitive adhesive preferably further contains a carboxylic acid ester. The carboxylic acid ester functions as an adjuster for pressure-sensitive adhesive strength that adjusts the pressure-sensitive adhesive strength of the second adhesive layer 43 by reducing the pressure-sensitive adhesive strength of the acrylic polymer. As the carboxylic acid ester, a carboxylic acid ester compatible with the acrylic polymer can be used. As the carboxylic acid ester, fatty acid triglyceride or the like can be used.

If necessary, the acrylic pressure-sensitive adhesive may contain a crosslinking agent. The crosslinking agent is a crosslinking component that crosslinks the acrylic polymer. Examples of the crosslinking agent include polyisocyanate compounds (polyfunctional isocyanate compounds), epoxy compounds, melamine compounds, peroxide compounds, urea compounds, metal alkoxide compounds, metal chelate compounds, metal salt compounds, carbodiimide compounds, oxazoline compounds, aziridine compounds, amine compounds, and the like. Of these, polyisocyanate compounds are preferable. These crosslinking agents may be used alone or in combination.

The second adhesive layer 43 preferably has excellent biocompatibility. For example, when the second adhesive layer 43 is subjected to a keratin peeling test, a keratin-peeled area percentage is preferably from 0% through 50%. When the keratin-peeled area percentage is in the range of from 0% through 50%, the burden on the skin 2 can be suppressed even if the second adhesive layer 43 is attached to the skin 2.

The second adhesive layer 43 preferably has moisture permeability. Water vapor and the like generated from the skin 2, to which the biological sensor 1 is attached, can be escaped toward the upper sheet 12 through the second adhesive layer 43. Also, as described below, the upper sheet 12 has a structure having cells. Thus, water vapor can be released to the exterior of the biological sensor 1 through the second adhesive layer 43. This can prevent sweat or water vapor from accumulating at the interface between the skin 2, to which the biological sensor 1 is attached, and the second adhesive layer 43. As a result, it is possible to prevent the adhesive strength of the second adhesive layer 43 from weakening due to the moisture accumulated at the interface between the skin 2 and the second adhesive layer 43, and prevent peeling of the biological sensor 1 off from the skin 2.

Preferably, the moisture permeability of the second adhesive layer 43 is, for example, from 300 g/(m²·day) through 10,000 g/(m²·day). When the moisture permeability of the second adhesive layer 43 is in the above preferable range, even if the second adhesive layer 43 is attached to the skin 2, sweat or the like generated from the skin 2 can be appropriately released toward the exterior through the second adhesive layer 43. This can reduce the burden on the skin 2.

The thickness of the second adhesive layer 43 can be appropriately selected, and is preferably from 10 μm through 300 μm. When the thickness of the second adhesive layer 43 is from 10 μm through 300 μm, the biological sensor 1 can become thinner.

As illustrated in FIGS. 1 and 2, when the biological sensor 1 is not in use, a release liner 50 is preferably attached to the surfaces of the electrode 20 and the second base 41 to be attached to the living body until use in order to protect the electrode 20 and the second layer member 40. Upon use, the release liner 50 is peeled off from the electrode 20 and the second layer member 40, and then the attachment surface of the biological sensor 1 can be attached to the skin 2. When the release liner 50 is attached to the attachment surface, the adhesive strength of the electrode 20 and the second layer member 40 can be maintained, for example, even if the biological sensor 1 is stored for a long time. Therefore, by peeling off the release liner 50 from the second layer member 40 and the electrode 20 upon use, the attachment surface can be reliably attached to the skin 2 for use.

No particular limitation is imposed on a production method of the biological sensor 1. However, the biological sensor 1 can be produced by any appropriate method. An example of the production method of the biological sensor 1 will be described.

The first layer member 10, the electrode 20, the sensor portion 30, and the second layer member 40 illustrated in FIGS. 1 and 2 are provided. No particular limitation is imposed on production methods of the first layer member 10, the electrode 20, the sensor portion 30, and the second layer member 40 as long as they can be produced. The first layer member 10, the electrode 20, the sensor portion 30, and the second layer member 40 can be produced by any appropriate methods.

After providing the first layer member 10, the electrode 20, the sensor portion 30, and the second layer member 40 that form the biological sensor 1 illustrated in FIG. 1, the sensor portion 30 is placed on the second layer member 40. Subsequently, the first layer member 10, the electrode 20, the sensor portion 30, and the second layer member 40 are stacked in the order from the first layer member 10 side toward the second layer member 40 side. In this manner, the biological sensor 1 illustrated in FIG. 1 is obtained.

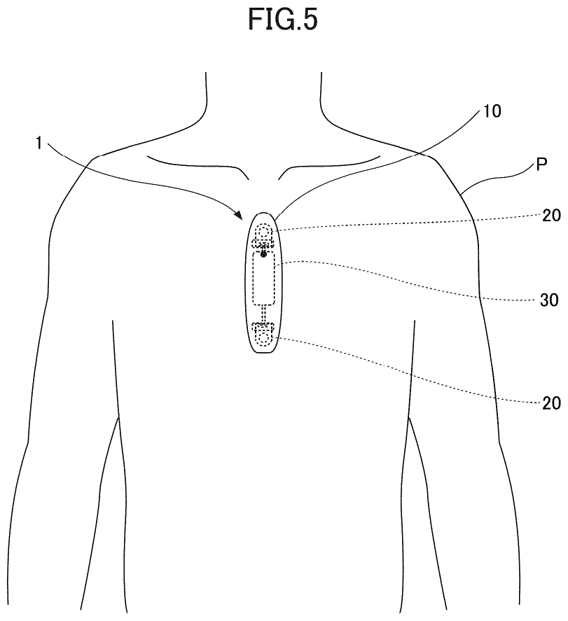

FIG. 5 is an explanatory view illustrating the biological sensor 1 of FIG. 1 attached to the chest of a subject P. As illustrated in FIG. 5, for example, the biological sensor 1 is attached to the skin of the subject P in a state in which the longitudinal direction (Y-axis direction) is aligned with the sternum of the subject P, and one electrode 20 faces upward and the other electrode 20 faces downward. When the biological sensor 1 is attached to the skin of the subject P by the effect of the second adhesive layer 43 of FIG. 2, the biological sensor 1 obtains biological signals, such as an electrocardiogram signal and the like, from the subject P via the electrode 20 in a state in which the electrode 20 is compressed to the skin of the subject P. The biological sensor 1 stores the obtained biological signal data in a non-volatile memory, such as a flash memory or the like, mounted on the part-mounting portion 321.

As described above, the biological sensor 1 includes the first layer member 10, the electrode 20, the sensor body 32, and the second layer member 40. The electrode 20 has a thickness of 15 μm or more, an area of from 2.0 cm² through 5.0 cm², and a tack force of 60 gf/Φ5 mm or more. Thus, the electrode 20 has an appropriate degree of flexibility and can be readily extended, such as, for example, extension of the biological sensor 1 in the longitudinal direction. This can enhance an attachment performance of the biological sensor 1 to the surface of the skin 2. Also, in the bottom view of the biological sensor 1, the covering percentage of the electrode 20 with respect to the first layer member 10 is from 40% through 90%. As such, the contact impedance of the biological sensor 1 can be reduced, and thus the generation of noise can be suppressed. Therefore, the biological sensor 1 can suppress the generation of noise during use, and can be stably attached to the living body.

Therefore, the biological sensor 1 can enhance the accuracy of the electrocardiogram waveform measured during measurement of an electrocardiogram as the biological signal, and can enhance the attachment performance to the surface of the skin 2.

According to the biological sensor 1, the first layer member 10 includes the first base 121 and the first adhesive layer 122, and the electrode 20 can be attached to the lower surface of the first adhesive layer 122, i.e., the surface on the second layer member 40 side. Because the first adhesive layer 122 has adhesiveness, the electrode 20 can contact the surface of the skin 2 in a state in which the electrode 20 is stably attached to the first layer member 10 via the first adhesive layer 122. This can further reduce the contact impedance of the electrode 20 with the surface of the skin 2, and can further suppress the generation of noise. Therefore, the biological sensor 1 can stably suppress the generation of noise during use, and can stably maintain the attachment performance to the living body.

The biological sensor 1 can include the second adhesive layer 43 on the surface of the second layer member 40 opposite to the first layer member 10. Thus, the second layer member 40 of the biological sensor 1 can be attached to the skin 2 via the second adhesive layer 43. This can further reduce the contact impedance of the electrode 20 with the surface of the skin 2, and can further suppress the generation of noise. Therefore, the biological sensor 1 can stably suppress the generation of noise during use, and can stably maintain the attachment performance to the living body.

The biological sensor 1 can form the attachment surface to the skin 2 by the first layer member 10, the electrode 20, and the second layer member 40. Thus, the thickness of the biological sensor 1 can be reduced. Therefore, the biological sensor 1 can be reduced in size, and can reduce the contact impedance with the surface of the skin 2.

According to the biological sensor 1, the electrode 20 can have adhesiveness. By virtue of the adhesiveness of the electrode 20, the electrode 20 can be attached to the lower surface of the first layer member 10 without providing the first layer member 10 with the first adhesive layer 122. Therefore, the thickness of the biological sensor 1 can be reduced. Also, the electrode 20 can be adhered to the skin 2, and thus the adhesiveness to the skin 2 can be maintained. Therefore, the biological sensor 1 can be further reduced in size, and can further effectively reduce the contact impedance with the surface of the skin 2.

Also, by virtue of the adhesiveness of the electrode 20, there is no need to provide the first layer member 10 with the first adhesive layer 122, and the biological sensor 1 can be reduced in size. Therefore, the production cost of the biological sensor 1 can be reduced.

Further, by virtue of the adhesiveness of the electrode 20, the electrode 20 can be adhered to the upper surface of the second layer member 40. Thus, the biological sensor 1 can reduce the contact resistance between the electrode 20 and the second layer member 40. Therefore, the biological sensor 1 can more stably detect the biological signals obtained from the skin 2.

According to the biological sensor 1, the electrode 20 includes a conductive polymer, a binder resin, and a moisturizer, and the binder resin can be formed of an aqueous emulsion adhesive. Thus, the electrode 20 can be lowered in resistance, and can enhance viscoelasticity and suppress swelling due to absorption of water. Therefore, the electrode 20 can enhance waterproofness, and thus can exhibit conductivity and adhesive strength, and can enhance flexibility and followability to the surface of the skin 2. As such, the electrode 20 can maintain adhesiveness to the skin 2, and thus the biological sensor 1 can more reliably reduce the contact impedance with the surface of the skin 2.

The biological sensor 1 can use an acrylic emulsion adhesive as the aqueous emulsion adhesive for the electrode 20. Therefore, the electrode 20 can be reliably enhanced in waterproofness, and thus can suppress a reduction in adhesive strength while maintaining resistance, and can reliably enhance followability to the surface of the skin 2. Therefore, the electrode 20 can be reliably reduced in viscoelasticity to a low level, and thus can have high adhesive strength and followability to the surface of the skin 2. As such, the biological sensor 1 can reliably reduce the contact impedance with the surface of the skin 2.

The biological sensor 1 can use, as the acrylic emulsion adhesive for the electrode 20, a silane-based emulsion adhesive that contains: a monomer mixture containing a (meth)acrylic acid alkyl ester; a water-dispersible copolymer obtained through copolymerization of a silane-based monomer copolymerizable with a (meth)acrylic acid alkyl ester; and an organic liquid component compatible with the water-dispersible copolymer. Thus, the electrode 20 can be reliably reduced in viscoelasticity to a low level, thereby enhancing the adhesive strength and further enhancing the followability to the surface of the skin 2. Therefore, the biological sensor 1 can further reliably reduce the contact impedance with the surface of the skin 2.

The biological sensor 1 can use, as the acrylic emulsion adhesive for the electrode 20, a two- or three-component acrylic emulsion adhesive containing one or more components selected from the group including: a monomer mixture containing a (meth)acrylic acid alkyl ester; and a carboxyl group-containing monomer mixture. In this case, the electrode 20 can be reliably reduced in viscoelasticity to a low level, thereby enhancing the adhesive strength and further enhancing the followability to the surface of the skin 2. Therefore, the biological sensor 1 can further reliably reduce the contact impedance with the surface of the skin 2.

As described above, the biological sensor 1 can stably measure biological information from the skin 2 during use for a long time. Thus, the biological sensor 1 can be effectively used as an attachable biological sensor that is attached in use to the skin 2 of a human or the like. For example, the biological sensor 1 exhibits high detection sensitivity of an electrocardiogram when attached to the skin of the living body or the like. Thus, the biological sensor 1 can be successfully used, for example, in a wearable device for health care that requires a high effect of suppressing noise generated in the electrocardiogram.

Although the embodiments have been described above, the above embodiments are merely illustrative, and the present invention is not limited to the above embodiments. The above embodiments can be practiced in various other forms, and various combinations, omissions, substitutions, changes, and the like can be made without departing from the gist of the invention. These embodiments and variations are encompassed in the scope and gist of the invention, and included in the scope equivalent to the inventions recited in the claims.

### EXAMPLES

The embodiments will be described in more detail with reference to Examples and Comparative Examples. However, the embodiments are not limited to these Examples and Comparative Examples.

### Example 1

### [Preparation of Electrode]

### (Preparation of Electrode 1)

0.8 g of a PEDOT/PSS pellet (Orgacon DRY, obtained from AGFA Materials Japan, LTD.) serving as a conductive polymer, 3.25 g of a silane-based emulsion adhesive (obtained from Nitto Denko Corporation) serving as a binder resin, and 1.6 g of glycerin (obtained from Wako Pure Chemical Industries, Ltd.) serving as a moisturizer were added to a plastic container. The mixture was stirred and defoamed using a planetary stirring apparatus, thereby preparing an adhesive electrode-forming composition that was homogeneous. The adhesive electrode-forming composition was removed and cured, thereby preparing a cured product of a conductive composition having adhesiveness. The cured product was punched (pressed) into a desired shape and formed into a sheet, thereby preparing an electrode 1, i.e., an adhesive electrode sheet (biological electrode). The thickness of the electrode 1 was 30 μm.

### (Preparation of Electrode 2)

### 1. Preparation of Conductive Composition

0.38 parts by mass of a PEDOT/PSS pellet (Orgacon DRY, obtained from AGFA Materials Japan, LTD.) serving as a conductive polymer, 10.00 parts by mass of an aqueous solution containing modified polyvinyl alcohol (modified PVA) (modified polyvinyl alcohol concentration: 10%, GOSENEX Z-410, obtained from The Nippon Synthetic Chemical Industry Co., Ltd.) serving as a binder resin, 2.00 parts by mass of glycerin (obtained from Wako Pure Chemical Industries, Ltd.) serving as a plasticizer, and 1.60 parts by mass of 2-propanol and 6.50 parts by mass of water serving as solvents were added to an ultrasonic bath. The aqueous solution containing these components was mixed in the ultrasonic bath for 30 minutes, thereby preparing an aqueous conductive composition solution A that was homogeneous.

The concentration of the modified PVA in the aqueous solution containing the modified PVA is about 10%, and thus the content of the modified PVA in the aqueous conductive composition solution A is 1.00 parts by mass. Note that the balance is the solvent in the aqueous conductive composition solution A.

The content of the conductive polymer, the content of the binder resin, and the content of the plasticizer per 100.0 parts by mass of the conductive composition were 11.2 parts by mass, 29.6 parts by mass, and 59.2 parts by mass, respectively.

### 2. Preparation of Electrode Sheet

The prepared aqueous conductive composition solution A was coated on a polyethylene terephthalate (PET) film using an applicator. Subsequently, the PET film coated with the aqueous conductive composition solution A was transferred to a dry oven (SPHH-201, obtained from ESPEC CORP.), and the aqueous conductive composition solution A was heated and dried at 135° C. for 3 minutes, thereby preparing a cured product of the conductive composition. The cured product was punched (pressed) into a desired shape and formed into a sheet, thereby preparing an electrode 2 that was an electrode sheet (biological electrode) having a thickness of 20 μm.

The content of the conductive polymer, the content of the binder resin, and the content of the plasticizer contained in the electrode 2 were similar to those in the conductive composition, and were 11.2 parts by mass, 29.6 parts by mass, and 59.2 parts by mass, respectively.

### [Tack Force of Electrode]

The tack force of the electrode was measured using a tacking tester (Tackiness Tester TAC1000, obtained from RHESCA CO., LTD.). As the measurement conditions, the load pressed on the electrode was 50 gf, the pressing speed was 0.01 mm/s, the retention time was 1.0 s, and the pulling speed was 1 mm/s. Table 1 illustrates the measurement results of the tack forces of the electrodes.

### [Preparation of Biological Sensor]

### (Preparation of Cover Member)

A cover member was prepared by forming a coat layer, having a Shore hardness of A40 and formed of silicone rubber, on a support formed using PET serving as a base resin, followed by molding into a predetermined shape.

### (Preparation of First Stacked Sheet)

A first adhesive layer was formed by attaching double-sided adhesive tape 1 (PKE-20, obtained from Nitto Denko Corporation, thickness: 60 μm) to the lower surface of a first base (polyolefin foamed sheet (FOLEC (registered trademark), obtained from INOAC CORPORATION, thickness: 0.5 mm) which was a porous base formed into a rectangular shape. The double-sided adhesive tape 1 is a double-sided adhesive tape in which an adhesive (acrylic resin) is formed on the surfaces. Subsequently, silicone tape (ST503 (HC) 60, obtained from Nitto Denko Corporation, thickness: 60 μm) was attached to the upper surface of the attachment layer to form an upper adhesive layer, thereby preparing a first stacked sheet.

### (Preparation of Second Stacked Sheet)

An adhesive (PERME-ROLL, obtained from Nitto Denko Corporation, moisture permeability: 21 g/(m²·day)) was adhered to both surfaces of a base formed into a rectangular shape (PET (PET-50-SCA1 (white), obtained from Mitsui & Co. Plastics Ltd.), thickness: 38 μm) to form a lower adhesive layer and a second adhesive layer, thereby preparing a second stacked sheet.

### (Preparation of Biological Sensor)

A sensor portion including a battery and a controller was placed in a center region of the upper surface of the second stacked sheet. Subsequently, adhesive electrodes, i.e., a pair of the electrodes 1, were attached to the attachment surface of the first adhesive layer of the first stacked sheet in a state of being held between the first adhesive layer and the second stacked sheet, thereby connecting the electrodes 1 to the interconnects of the sensor portion. Subsequently, the cover member was stacked on the first stacked sheet such that the sensor portion was disposed in a housing space formed by the first stacked sheet and the cover member, thereby preparing a biological sensor.

### (Adhesive Strength of First Adhesive Layer)

After attaching backing tape to one surface of the adhesive, a sample piece (10 mm in width×50 mm in length) was cut out from the backing tape-attached adhesive sheet. Next, the surface of the sample piece on the adhesive sheet side was attached to a resin plate (Bakelite board) using a laminator. Then, using a tensile tester (Autograph AGS-50NX, obtained from Shimadzu Corporation), a peeling test of pulling the backing tape of the sample piece on the resin plate was performed under the conditions of 23° C. in temperature, 180° in a peeling angle, and 300 mm/min in a peeling speed, thereby measuring the peeling-resistant adhesive strength (peel-resistant force) (unit: N/10 mm) of the adhesive sheet to the resin plate at 23° C.

### (Area of First Adhesive Layer)

Images of the bottom surface of the prepared biological sensor were obtained. Of the images of the bottom surface of the biological sensor, the image of the bottom surface of the first adhesive layer was binarized using image analysis software (ImageJ), and an area S1 of the first adhesive layer expressed in dots (pixels) was calculated.

### (Area of Electrode)

Images of the bottom surface of the prepared biological sensor were obtained. Of the images of the bottom surface of the biological sensor, the image of the bottom surface of the electrode was binarized using image analysis software (ImageJ), and an area S2 of the electrode expressed in dots (pixels) was calculated.

### (Covering Percentage of Electrode)

A covering percentage of the electrode was obtained from the following formula (1) using the above-obtained area S1 of one first adhesive layer and the above-obtained area S2 of one electrode.

$$Covering Percentage of Electrode ( \% ) = Area S 2 / ( Area S 1 + Area S 2 ) \times 100 \qquad ( 1 )$$

The measurement results of the area of the first adhesive layer, the area of the electrode, and the covering percentage and the tack force of the electrode are illustrated in Table 1.

### Example 2

A biological sensor was prepared in the same manner as in Example 1 except that unlike in Example 1, double-sided adhesive tape 2 (H-PAO, obtained from Nitto Denko Corporation, thickness: 60 μm) was used as the first adhesive layer instead of the double-sided adhesive tape 1, and the area S1 of one first adhesive layer and the area S2 and the covering percentage of one electrode were changed as illustrated in Table 1.

### Comparative Example 1

A biological sensor was prepared in the same manner as in Example 1 except that unlike in Example 1, the electrode 1 was changed to the electrode 2, and the area S1 of one first adhesive layer and the area S2 and the covering percentage of one electrode were changed as illustrated in Table 1.

### Comparative Example 2

A biological sensor was prepared in the same manner as in Example 1 except that unlike in Example 1, double-sided adhesive tape 2 (PH-PAO, obtained from Nitto Denko Corporation, thickness: 60 μm) was used as the first adhesive layer instead of the double-sided adhesive tape 1, and the area S1 of one first adhesive layer and the area S2 and the covering percentage of one electrode were changed as illustrated in Table 1.

The measurement results of the area of the first adhesive layer, the area of the electrode, and the covering percentage and the tack force of the electrode in the above Examples and Comparative Examples are illustrated in Table 1.

### <Evaluation of Characteristics of Biological Sensor>

The waveform accuracy and the attachment performance of the biological sensor in the Examples and Comparative Examples were measured and evaluated.

### [Waveform Accuracy]

The waveform accuracy of the biological sensor was evaluated in accordance with the following evaluation criteria by measuring the contact impedance when attaching the biological sensor to a subject for 24 hours and measuring an electrocardiogram. When the contact impedance is 400 kΩ or less, the biological sensor is effectively used at the time of measurement of an electrocardiogram.

### (Evaluation Criteria)

A: The contact impedance is 400 kΩ or less.
B: The contact impedance is more than 400 kΩ.

### [Attachment Performance]

The attachment performance of the biological sensor was evaluated in accordance with the following evaluation criteria when attaching the biological sensor to a subject for 24 hours and measuring an electrocardiogram.

### (Evaluation Criteria)

A: The biological sensor remains attached to the skin of the subject.
B: At least a part of the biological sensor is peeled off from the skin of the subject.

TABLE 1

| | First adhesive layer | | | | | | | | | |
|---|---|---|---|---|---|---|---|---|---|---|
| | | Adhesive | | Electrode | | | | | | |
| | | strength | | | | | Area | | | |
| | | [N/10 mm] | | | | | (one | Covering | Biological sensor | |
| | | (on Bakelite | Area | | Self- | Tack force | electrode) | percentage | | Attachment |
| | Type | board) | [cm²] | Type | adhesiveness | [gf: Φ5 mm] | [cm²] | [%] | Noise | performance |
| Example 1 | Double-sided | 7.3 | 1.8 | Electrode 1 | Present | 66.0 | 3.9 | 68.0 | A | A |
| | adhesive tape 1 | | | | | | | | | |
| Example 2 | Double-sided | 5.0 | 3.0 | Electrode 1 | Present | 66.0 | 2.7 | 47.0 | A | A |
| | adhesive tape 2 | | | | | | | | | |
| Comparative | Double-sided | 7.3 | 4.1 | Electrode 2 | Absent | 3.7 | 1.6 | 28.0 | B | B |
| Example 1 | adhesive tape 1 | | | | | | | | | |
| Comparative | Double-sided | 5.0 | 4.3 | Electrode 2 | Absent | 3.7 | 1.4 | 25.0 | B | B |
| Example 2 | adhesive tape 2 | | | | | | | | | |

From Table 1, it was confirmed in Examples 1 and 2 that the biological sensor satisfied the requirements for use in both of the waveform accuracy and the attachment performance. On the other hand, it was confirmed in Comparative Examples 1 and 2 that at least one or more of the waveform accuracy and the attachment performance of the biological sensor did not satisfy the requirements for use.

Therefore, the biological sensor of each of the Examples, in which the thickness, the area, and the tack force of the electrodes were set to be equal to or less than respective predetermined values, was able to suppress the noise generated in the electrocardiogram during the measurement of the electrocardiogram, i.e., stably obtain the electrocardiogram waveform, and also was able to stably maintain the state of attachment to the skin of the subject, i.e., maintain the attachment performance. Therefore, even if the biological sensor according to the present embodiment is attached to the skin of a subject for a long time (e.g., 24 hours), the biological sensor can be effectively used to measure an electrocardiogram continuously for a long time.

Embodiments of the present invention are, for example, as follows.
<1> A biological sensor, including:
   a sensor body configured to obtain biological information;
   an electrode having adhesiveness and connected to the sensor body;
   a first layer member including a housing space in which the sensor body is housed, the electrode being disposed on a lower surface of the first layer member; and
   a second layer member that is attached to the lower surface of the first layer member so as to expose the electrode and cover the sensor body, in which
   a thickness of the electrode is 15 μm or more, an area of the electrode is from 2.0 cm² through 5.0 cm², and a tack force of the electrode is 60 gf/Φ5 mm or more, and
   in a bottom view, a covering percentage of the electrode with respect to the first layer member is from 40% through 90%.
<2> The biological sensor according to <1>, in which
   the first layer member includes
      a first base including a through-hole at a position corresponding to the housing space, and
      a first adhesive layer that is provided at a surface of the first base, the surface of the first base facing a living body, and to which the electrode is attached, and
   the electrode is attached to a surface of the first adhesive layer, the surface of the first adhesive layer facing the second layer member.
<3> The biological sensor according to <1> or <2>, in which
   the second layer member includes
      a second adhesive layer at a surface opposite to the first layer member.
<4> The biological sensor according to any one of <1> to <3>, in which
   an attachment surface to a living body is formed by the first layer member, the electrode, and the second layer member.

The present application claims priority to Japanese Patent Application No. 2022-090963, filed on Jun. 3, 2022 with the Japan Patent Office, and the entire contents of the above application are incorporated herein by reference.

### REFERENCE SIGNS LIST

1 Biological sensor
2 Skin
10 First layer member
11 Cover member
12 Upper sheet
12a, 121a, 122a Through-hole
20,20A, 20B Electrode
30 Sensor portion
31 Flexible substrate
32 Sensor body
33A Connection portion
33A, 33B Connection portion
34 Battery
40 Second layer member
41 Second base
42 Lower adhesive layer
43 Second adhesive layer
111 Projection
111a Recess
112A, 112B Flat portion
121 First base
122 First adhesive layer
123 Upper adhesive layer
201A, 201B Facing portion
202A, 201B Exposed portion
321 Part-mounting portion
322 Battery-mounting portion
331A, 331B Interconnect
332A, 332B Terminal

1. A biological sensor, comprising:
a sensor body configured to obtain biological information;
an electrode having adhesiveness and connected to the sensor body;
a first layer member including a housing space in which the sensor body is housed, the electrode being disposed on a lower surface of the first layer member; and
a second layer member that is attached to the lower surface of the first layer member so as to expose the electrode and cover the sensor body, wherein
a thickness of the electrode is 15 μm or more, an area of the electrode is from 2.0 cm² through 5.0 cm², and a tack force of the electrode is 60 gf/Φ5 mm or more, and
in a bottom view, a covering percentage of the electrode with respect to the first layer member is from 40% through 90%.

2. The biological sensor according to claim 1, wherein
the first layer member includes
a first base including a through-hole at a position corresponding to the housing space, and
a first adhesive layer that is provided at a surface of the first base, the surface of the first base facing a living body, and to which the electrode is attached, and
the electrode is attached to a surface of the first adhesive layer, the surface of the first adhesive layer facing the second layer member.

3. The biological sensor according to claim 1, wherein
the second layer member includes
a second adhesive layer at a surface opposite to the first layer member.

4. The biological sensor according to claim 1, wherein
an attachment surface to a living body is formed by the first layer member, the electrode, and the second layer member.

5. The biological sensor according to claim 2, wherein
an attachment surface to a living body is formed by the first layer member, the electrode, and the second layer member.

6. The biological sensor according to claim 3, wherein
an attachment surface to a living body is formed by the first layer member, the electrode, and the second layer member.
